(19) United States
(12) Patent Application Publication
(10) Pub. No.: US 2007/0082008 A1
(43) Pub. Date: Apr. 12, 2007

(54) Feed formulation for terrestrial and aquatic animals

(75) Inventors: Moti Harel, Baltimore, MD (US); Diane Clayton, Hauteville (CH); Robert Alan Bullis, Ellicott City, MD (US)

Correspondence Address: DUANE MORRIS, LLP;IP DEPARTMENT, 30 SOUTH 17TH STREET, PHILADELPHIA, PA, 19103-4196, US

(73) Assignee: Advanced Bionutrition Corporation, Columbia, MD (US)

(21) Appl. No.: 10/548,363

(22) PCT Filed: Mar. 5, 2004
(86) PCT No.: PCT/US04/05223

§ 371 Date: Dec. 20, 2006

Related U.S. Application Data
(60) Provisional application No. 60/452,529, filed on Mar. 7, 2003.
(60) Provisional application No. 60/510,537, filed on Oct. 14, 2003.

(51) Int. Cl. A61K 36/06; A61K 36/02; A23K 1/165

(52) U.S. Cl. 424/195.16; 424/442; 424/195.17; 426/635

(57) ABSTRACT

The use of macroalgal, microalgal, and fungally-derived materials provide, in combination with higher-plant derived materials, complete feeds for animal husbandry. The products and methods of the invention provide nutritional feed formulations, that reduce or eliminate the need for animal-derived materials. The feeds are useful for terrestrial or aquatic animals, and comprise docosahexaenoic acid and eicosapentaenoic acid.

### PRIORITY CLAIM

This application claims the priority of provisional application 60/452,529, filed in the United States Patent and Trademark Office on Mar. 7, 2003 and provisional application 60/510,537, filed in the United States Patent and Trademark Office on Oct. 14, 2003.

### BACKGROUND OF THE INVENTION

Animal-derived by-products and meals are currently added to feed formulations for both terrestrial and aquatic animals. The rates of usage of animal-derived by-products and meals vary from a few percent to twenty five percent of the total feed. Reliance on animal by-products to deliver essential amino acids, vitamins, oils and other compounds is dangerous both to humans and the environment. They can directly affect human health, for example with manifestations as problems with disease transmission (such as mad cow disease) have demonstrated. Prions and other disease causing agents are capable of surviving processing, and entering into the animal being fed an animal-derived meal. Humans consuming such an animal's meat are subject to diseases such as the new variant Creutzfeld-Jacob Disease (nvCJD).

Reliance on animal products can also have a detrimental effect on public heath globally. For example, the use of fishmeal and fish oil has devastated some fish fisheries that produce fish deemed undesirable for various reasons, but useful in the production of fish oil and fishmeal. This fish oil and fishmeal serves to feed other fish, and the oceans are being thrown out of balance by the widescale harvest of fish for use as the use as fishmeal and fish oil.

One example of an animal-derived meal being extensively used in feeds is fishmeal. Fishmeal is currently being added to a substantial portion of both terrestrial and aquatic animal feeds. Most terrestrial and aquaculture animal diets are based on a mixture of plant meals (soy, corn, wheat, and etc.) and animal meals (meat meal, blood meal, bone meal, fishmeal, and/or fish oil). The animal-derived meals provide both highly digestible proteins as well as essential long chain fatty acids. Fishery-based products are particularly beneficial because of their unique balance of protein (amino acids) and lipids (long chain omega-3 fatty acids) in a highly digestible, energy dense form. Although a considerable amount of work has been performed with the goal of developing substitutes for fishmeal and fish oil with products like soy and wheat, a high level of replacement has been unsuccessful. This is not surprising, given the balance of nutrients and their natural role, since fishmeal and fish oil are produced metabolically from fish, or are acquired from the complex natural food chain. Substitution with other ingredients, especially those of vegetable origin, is likely to be inadequate in protein content and digestibility. Likewise, in terrestrial agriculture, fishmeal supplementation improves the nutritional status of the animal, and delivers both health and welfare benefits.

One specific benefit of the protein component of fishmeal is its digestibility. Fishmeal also has a high level of essential amino acids such as lysine, threonine and tryptophan, as well as the sulfur-containing amino acids methionine and cysteine. Proteins from cereal grains and most other plant protein concentrates fail to supply complete amino acid needs primarily due to a shortage of methionine and/or lysine. Soybean meal, for example, is a good source of lysine and tryptophan, but it is low in the sulfur-containing amino acids methionine and cysteine. The essential amino acids in fishmeal are also in the form of highly digestible peptides. Plant and cereal proteins generally are not in such as highly digestible form, and are also accompanied by indigestible fiber. In addition to its protein component, fishmeal also has a relatively high content of certain minerals, such as calcium and phosphorous, as well as certain vitamins, such as B-complex vitamins (e.g., choline, biotin and B12), and vitamins A and D. Industrial fishmeal usually also contains about 15% fish oil, which provides a source of important essential fatty acids.

Specific benefits of fish oil include providing certain lipid-soluble vitamins (e.g., Vitamin A from fish liver oils) and certain preformed long chain polyunsaturated fatty acids (LC-PUFAs), such as arachidonic acid (ARA), eicosapentaenoic acid (EPA), and docosahexaenoic acid (DHA) (see FIG. 1). These LC-PUFAs are not produced by conventional plant sources (such as soy, corn, palm, canola, etc.) and are generally provided in feeds in small quantities by the provision of animal products. Fish oil is particularly rich in these compounds. Other animal sources of LC-PUFAs include animal offal and/or process by-products (e.g., blood meal, organ meats, etc), egg-based products, and invertebrates (e.g. polychetes, crustaceans, insects and nematodes).

LC-PUFAs are a required component of many diets because of their essentiality in optimnum cellular and metabolic functions. Neurological tissues, for example, are highly enriched in DHA and ARA. The fatty acid precursors of DHA and ARA are linolenic acid (ALA) and linoleic acid (LA), respectively, and are generally considered essential nutrients in animal diets because of a metabolic inability to produce these fatty acids de novo. Most animals can elongate and desaturate precursors to the LC-PUFAs essential for optimal growth and development but their ability to do so is limited. Consequently, optimal growth and development usually accompanies dietary supplementation of the preformed LC-PUFAs (such as ARA, EPA, and DHA). Thus, nutritional feeds for animals typically contain these preformed LC-PUFAs as delivered by fish oil. These components are also supplied by conventional fishmeal, since fishmeal typically contains about 15% fish oil. Many researchers believe that one of the major benefits of fishmeal comes from the supply of fish oil associated with the fishmeal (and thus LC-PUFAs).

The increasing demand for fishmeal and fish oil combined with decreasing wild fish stocks indicate that an alternative product (or products) would be highly desirable. Fishmeal production over the last decade has fluctuated between 6.3 and 7.4 million metric tons (MMT) per year, while fish oil production has ranged between 1.0 and 1.7 MMT. The poultry industry uses 24%, pigs 29%, farmed fish 35%, and ruminants 3% of the total global fishmeal being consumed. With anticipation of a major increase in the production of both fish and chicken, global fishmeal requirements are projected to double by 2010. Shortly thereafter, it is predicted that aquaculture alone would be able to consume all the available fishmeal and fish oil production. Besides ecological and ethical opposition to the use of finite and valuable aquatic resources as feed ingredients for high value animal species, there is a growing economical concern about the uncertain availability and cost. An additional reason for concern is that fishery products may contain toxic compounds, as many fishing grounds have become increasingly contaminated with industrial pollution (e.g., mercury, PCBs, dioxin, mycotoxins, pesticides etc.). Consequently, industries that use fishmeal will be eventually forced to find alternatives, which are of high quality, nutritionally equivalent, and sustainable. In particular, European current agricultural practice is moving towards non-animal delivery forms of key nutrients, such as the n-3 fatty acids for terrestrial animal feeds (poultry, swine). Furthermore, for ruminant species, the use of animal meal and fishmeal is now prohibited in several countries (U.K. Dept. of Environment Agency for Food Standards report ACAF/01/38).

Formulating an animal feed based on increased growth rate and improved feed conversion is a driving principle for the feed formulator. In order to maintain the quality of the final diet, greater demand will be made on the quality of the meal ingredients. One answer to this problem may be production of microbial biomass through biotechnology. Newly developed methods of algal, yeast, and bacterial fermentation show promise for the development of superior sources of proteins and oils for use in formulated feeds. The huge variety of algae species (both macro- and microalgae), with their very diverse production of useful biomolecules could supply nutritional qualities.(e.g., essential amino acids, fatty acids, vitamins, minerals and secondary metabolites) to the meal industry that has not been fully utilized. In addition, the consumer's perception on what is safe, natural, and environmentally friendly will increasingly dominate future feed formulation decisions.

Macroalgae have been used as part of the feeds for domestic animals (Adey and Purgason 1998; Simopoulos 1999; GS et al. 2000; He et al. 2002). For the most part, the macroalgae have enjoyed most support for their high content of trace elements (e.g., iodine), essential vitamins (e.g., Vitamins B, D & E), antioxidants (e.g., carotenoids), and phytohormones. Macroalgae have recently been added to mammalian and poultry feeds as immunoenhancers to increase mammal and poultry resistance to disease (Allen and Pond 2002; Allen et al. 2002). Both macroalgal meals and extracts were shown to enhance the immune responses of mammals and poultry when used to supplement the diet. Macroalgae are generally collected from the sea or grown in nets in the ocean.

Microalgae have been used less extensively as a feed ingredient; the major microalga that is used is actually a cyanobacterium (also known as bluegreen algae). This cyanobacterium, Spirulina platensis, has been cultivated extensively and potentially provides health benefits to certain animals (Grinstead et al. 2000; Lu et al. 2002). Microalgae have also been utilized for their pigments (Abe et al. 1998; Ginzberg et al. 2000) and fatty acids in animal feeds (Simopoulos 1999). Microalgae are a very diverse group of organisms that produce interesting bioactive compounds, vitamins, hormones, essential amino acids, fatty acids, and etc. Pharmaceutical companies have been mining the microalga kingdom for bioactive compounds for the last twenty years or more. Additionally, microalgae have the advantage of enclosed growth (i.e., photobioreactors or fermentors) that is predictable, of assured quality, and a renewable resource. Recent advances in microalgal heterotrophic growth technology have advanced production of microalgae in standard fermentors to an economical method of production (Boswell et al. 1992; Behrens and Kyle 1996; Kyle et al. 1998).

Other microbial sources of LC-PUFAs include lower plants or fungi. These have been used even less extensively as feeds. Fungal species of the genus Mortierella have been used as a source of LC-PUFA-containing oils and have been cultivated in commercial scale fermentors for the production thereof. However, neither the fungal meal nor the whole fungi have been contemplated for use as a feed ingredient.

Thus, there is a need for new methods to reduce or eliminate the use of animal-based meals or by-products in feeds for terrestrial and aquatic organisms.

The inventors have discovered a method and a product that will provide optimal growth to aquatic and terrestrial animals without the need for introduction of animal by-products into the feed. Existing feeds often require the use of animal-derived meals or extracts to supply essential factors to the animal feed. Plant based feeds are appropriate for some animal species, however, a large number of animals raised in captivity require materials that are especially high in animal products. One example of compounds supplied in animal-derived materials are the omega three fatty acids and lipids high in levels of long-chain omega three fatty acids. Specific sterols are essential for the growth of specific animals, such as shrimp, which must have cholesterol in their diets. The abundance of fish in the oceans in the past has led to a reliance on the use of marine fishmeals, fish oil, and fish by-products for both terrestrial and aquatic animal feeds. Animal meals and by-products from meat processing and rendering plants have long been utilized as cheap and nutrient-rich (especially high lipids and protein) ingredients for animal husbandry.

Recent developments in the United Kingdom and elsewhere have cast doubt on the safety of the utilization of animal products in animal feeds destined for human consumption. Transfer of infectious agents to the animal being fed, a very real danger with the spread of bovine spongioform encephalitis (BSE), new variant Creutzfeld-Jacob Disease (nCJD), viral diseases (e.g., white spot virus, WSV), and other diseases, have been proven refractory to destruction by processing.

Additionally, the current dependence of fishmeal and fish oil has resulted in environmental damage by destruction of wild fisheries used by the higher food chain predatory fish (and cetaceans) that has resulted in catastrophic decreases in ocean productivity. Therefore, the invention described herein provides a novel approach to a real and pressing problem.

### SUMMARY OF THE INVENTION

It is an object of the invention to provide a feed composition, wherein all animal-derived components have been eliminated and microalgae, macroalgae, plants, and/or lower fungi, including extracts or components thereof, are included in the feed.

It is an object of the invention to provide a feed composition, wherein animal-derived components have been substantially eliminated and microalgae, macroalgae, plants, and/or lower fungi, including extracts or components thereof, are included in the feed.

It is an object of the invention to provide a method for preparation of an aquatic or terrestrial animal feed comprising a composition wherein all animal-derived components have been eliminated and microalgae, macroalgae, plants, and/or lower fungi, including extracts or components thereof, are included in the feed.

It is an object of the invention to provide a method for aquatic or terrestrial animal husbandry using a feed composition wherein all animal-derived components have been eliminated by the addition of microalgae, macroalgae, plants and/or lower fungi and yeast, including combinations thereof, in such a way to provide optimal growth without addition of animal-derived materials.

The current invention utilizes the broad nutritional potential of biomass from members of the algal kingdom in combination with plants and/or members of the lower fungi to adequately provide essential nutrients to feed formulations such that the need for animal-derived materials is either completely or substantially eliminated.

The invention provides an animal feed comprising macroalgae-derived materials, wherein no animal-derived materials are present. The macroalgae-derived materials can comprise from about 0.1% to about 30% of the dry weight of the feed. This feed can comprise from about 0.25% to about 5% combined DHA and EPA. The macroalgae-derived materials in this feed can comprise bioactive compounds. The bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention also provides an animal feed comprising microalgae-derived materials, wherein no animal-derived materials are present. These microalgae-derived materials can comprise from about 0.1% to about 30% of the dry weight of the feed. This feed can comprise from about 0.25% to about 5.0% combined DHA and EPA. The microalgae-derived materials comprise bioactive compounds. Their bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention further provides an animal feed comprising lower fungi-derived materials, wherein no animal-derived materials are present. The lower fungi-derived materials can comprise from about 0.1% to about 30% of the dry weight of the feed. This feed can comprise from about 0.25% to about 5.0% combined DHA and EPA. The lower fungi-derived materials can comprise bioactive compounds. Their bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention further provides an animal feed comprising plant-derived materials, wherein no animal-derived materials are present. The plant-derived materials can comprise from about 0.1% to about 30% of the dry weight of the feed. This feed can comprise from about 0.25% to about 5.0% combined DHA and EPA. The plant-derived materials can comprise bioactive compounds. Their bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention yet further provides animal feed comprising macroalgae-derived, microalgae-derived, plant, and/or lower fungi-derived materials, wherein no animal-derived materials are present. The macroalgae-derived, microalgae-derived, plant-derived, and/or lower fungi-derived materials can comprise from about 0.1% to about 30% of the dry weight of the feed. The feed can comprise from about 0.25% to about 5.0% combined DHA and EPA. This macroalgae-derived microalgae-derived, plant-derived, and/or lower fungi-derived materials can comprise bioactive compounds. The bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention provides an animal feed comprising macroalgae-derived materials and less than about 5% animal-derived materials. It can further comprise from about 0.25% to about 5.0% combined DHA and EPA. The macroalgae-derived materials can comprise from about 0.1% to about 30% of the dry weight of the feed. These macroalgae-derived materials can comprise bioactive compounds. Their bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention also provides an animal feed comprising microalgae-derived materials and less than about 5% animal-derived materials. The microalgae-derived materials can comprise from about 0.1% to about 30% of the dry weight of the feed. The feed can further comprising from about 0.25% to about 5.0% combined DHA and EPA. The microalgae-derived materials comprise bioactive compounds. Their bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention further provides an animal feed comprising lower fungi-derived materials and less than about 5% animal-derived materials. The lower fungi-derived materials can comprise from about 0.1% to about 30% of the dry weight of the feed. The feed can further comprise from about 0.25% to about 5.0% combined DHA and EPA. The lower fungi-derived materials can comprise bioactive compounds. Their bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention further provides an animal feed comprising plant-derived materials and less than about 5% animal-derived materials. The plant-derived materials can comprise from about 0.1% to about 30% of the dry weight of the feed. The feed can further comprise from about 0.25% to about 5.0% combined DHA and EPA. The plant-derived materials can comprise bioactive compounds. Their bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention yet further provides an animal feed comprising macroalgae-derived, microalgae-derived, plant-derived, and/or lower fungi-derived materials and less than about 5% animal-derived materials. The macroalgae-derived, microalgae-derived, plant-derived, and lower fungi-derived materials can comprise from about 0.1% to about 30% of the dry weight of the feed, which can further comprise from about 0.25% to about 5.0% combined DHA and EPA. The macroalgae-derived, microalgae-derived, and/or lower fungi-derived materials can comprise bioactive compounds. Their bioactivity can be chosen from one or more of immunoenhancement, growth promotion, disease resistance, antiviral action, antibacterial action, improved gut function, probiont colonization stimulation, improved food conversion, improved reproductive performance, and improved coat or skin.

The invention provides an animal feed or feed additive comprising a plant-derived material comprising DHA, EPA, or ARA, but no animal-derived materials. It also provides an animal feed or feed additive comprising a plant-derived material comprising DHA, EPA, or ARA, wherein animal-derived materials are present. The animal-derived materials can be poultry by-product meal, and can comprise from about 1% to 5% of the total feed. The plant-derived material can be derived from a plant comprising DHA, EPA, or ARA. The plant can be genetically modified.

The invention provides a method of preparing a feed comprising from about 0.25% to about 5.0% combined DHA and EPA, and further comprising materials derived from macroalgae, microalgae, plants, and/or lower fungi or any parts or extracts thereof, wherein no animal-derived materials are present.

The invention also provides a method of preparing a feed comprising from about 0.25% to 5.0% combined DHA and EPA, and further comprising materials derived from macroalgae, microalgae, plants, and/or lower fungi and/or any parts or extracts thereof, wherein less than about 5% animal-derived materials are present.

The invention further provides a method of feeding animals with a feed comprising from about 0.25% to about 5.0% combined DHA and EPA, materials derived from macroalgae, microalgae, plants, and/or lower fungi and/or any parts and/or extracts thereof, wherein no animal-derived materials are present.

The invention yet further provides a method of feeding animals with a feed comprising from about 0.25% to about 5.0% combined DHA and EPA, materials derived from macroalgae, microalgae, plants, and/or lower fungi and/or any parts and/or extracts thereof, and further comprising less than about 5% animal-derived materials.

The invention provides a method of preparing an animal feed or feed additive comprising a plant-derived material comprising DHA, EPA, or ARA, but no animal-derived materials. It also provides a method of preparing an animal feed or feed additive comprising a plant-derived material comprising DHA, EPA, or ARA, wherein animal-derived materials are present. The animal-derived materials can be poultry by-product meal, and can-comprise from about 1% to 5% of the total feed. The plant-derived material can be derived from a plant comprising DHA, EPA, or ARA. The plant can be genetically modified.

The invention also provides a method of feeding animals with a feed or feed additive comprising a plant-derived material, comprising DHA, EPA, OR ARA, but no animal-derived materials. It also provides a method of feeding animals with a feed or feed additive comprising a plant-derived material comprising DHA, EPA, OR ARA, wherein animal-derived materials are present. The animal derived materials can be poultry by-product meal, and can comprise from about 1% to about 5% of the total feed. The plant-derived material can be derived from a plant comprising DHA, EPA, or ARA. The plant can be genetically modified.

### BRIEF DESCRIPTION OF THE DRAWING

FIG. 1. Omega-3 and Omega-6 Fatty Acid Biochemical Pathways. Fatty acids are designated by the number of carbons followed by the number of double bonds. Also listed are typical sources for certain fatty acids. The following abbreviations are used: linoleic acid (LA), gamma linolenic acid (GLA), dihomo-gamma linoleic acid (DGLA), arachidonic acid (ARA), alpha linolenic acid (ALA), eicosapentaenoic acid (EPA) and docosahexaenoic acid (DHA).

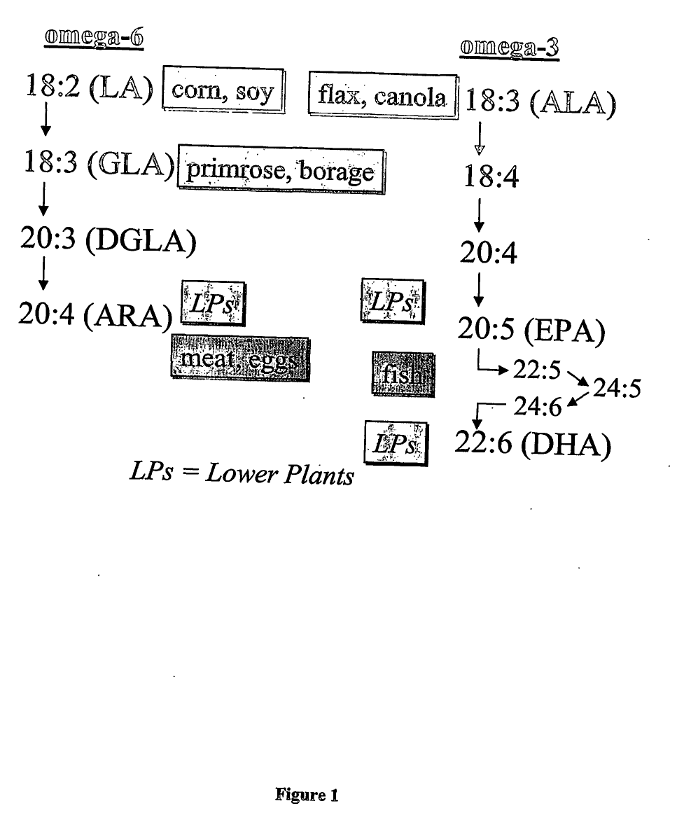

### DETAILED DESCRIPTION OF THE INVENTION

Definitions

In describing the present invention, the following terminology is used in accordance with the definitions set out below.

The term “animal feed” refers to a preparation providing nutritional value to any animal, including but not limited to terrestrial animals (humans, cattle, horses, pigs, sheep, goats, poultry) and aquatic animals (fish, shrimp, lobsters, crawfish, mollusks, sponges, jellyfish).

The term “fishhmeal” is used to describe a crude preparation or hydrolysate from fish of any species or mixed species that is processed into a solid or semi-solid form for easy use.

The term “fish oil” refers to any oil extracted from fish, in any form and purity. Usually in feed terms, “fish oil” is used to describe a fairly crude preparation but can also encompass a highly purified form used as a human food supplement.

The term “animal meal” is used to as a group descriptor to include fishmeal, meat meal, blood meal, beef extracts, and other animal-derived feed supplements.

The term “animal-derived” is used to describe any product produced from animals.

The term “probiont” refers to an organism that permanently or transiently grows or resides in the intestine of the target animal.

The term “macroalgae” refers to algae that in at least one life stage form large structures that are easily discernable with the naked eye. Usually these organisms have secondary vascularization and organs. Examples of different groups containing macroalgae follow, but are not limited to, the chlorophyta, rhodophyta, and phaeophyta. “Macroalgae-derived” materials are those that are obtained from macroalgae.

The term “microalgae” refers to prokaryotic and eukaryotic algae that are classed in many different species. Normally the prokaryotic algae are referred to as cyanobacteria or bluegreen algae. The eukaryotic microalgae come from many different genera, some of which overlap with the macroalgae and are differentiated from these by their size and a lack of defined organs (although they do have specialized cell types). Examples of different groups containing microalgae follow, but are not limited to, chlorophyta, rhodophyta, phaeophyta, dinophyta, euglenophyta, cyanophyta, prochlorophyta, and cryptophyta. “Microalgae-derived” materials are those that are obtained from microalgae.

The term “lower fungi” refers to fungi that are typically grown in fermentors by providing appropriate carbon and nitrogen sources. Examples of such lower fungi include, but are not limited to, yeasts (e.g., Saccharomyces, Phaffia, Pichia, and etc.), filamentous fungi (Mortierella, Saprolegnia, Pythium, and etc.), and chytrids (Schizochiytrium, Thraustochytriumn, Ulkenia, and etc.)

The terms “feed additive,” “food supplement,” or “enrichment product” refer to products having one or more nutritional substances in concentrated form (mainly vitamins, minerals and trace elements), usually presented in formats that are added to a complete diet or added separately as tablets, pellets, or beads to be consumed directly. Feed additives, food supplements, or enrichments are not meant to fullfill the complete needs of the animal but provide some specific benefit. For the purposes herein the two terms will be used synonomously.

The present invention is related to a composition of algal and/or fungal mixtures for use as an ingredient in complete non-animal based feeds. These feeds could also provide improved growth food conversion ratios, survival rate and health of terrestrial and aquatic animals since many macro- and microalgae and fungi have demonstrated bioactivities (Mason and Gleason 1981; Metting and Pyne 1986; Jones 1988; De Rosa et al. 2001; Kumvan et al. 2001; Neves et al. 2001; Oufdou et al. 2001; Faullner 2002; Gonzalez et al. 2002; Hellio et al. 2002; Minton et al. 2002; Piccardi et al. 2002; Prati et al 2002; Seya et al. 2002; Tan and Siddiq 2002).

These and other aspects of the invention are provided by one or more of the following embodiments.

One embodiment of the invention is a feed or feed ingredient wherein all animal products are eliminated and the feed contains a macroalgal biomass, macroalgal cells, or macroalgal derivatives comprising materials from one or more macroalgal species selected from, but not limited to, the following organisms, Laminaria, Padina, Pavonica, Gracilaria, Viva, and Ascophyllum.

Another embodiment of the invention is a feed or feed ingredient wherein all animal products are eliminated and the feed contains a microalgal biomass, microalgal cells, or microalgal derivatives comprising materials from a one or more species selected from, but not limited to, the following organisms, Crypthecodinium, Tetraselmis, Chlorella, Haematococcus, Nitzschia, Chaetoceros, Spirulina, and Arthrospiria.

Another embodiment of the invention is a feed or feed ingredient wherein all animal products are eliminated and the feed contains a lower fungal biomass, lower fungal whole cells, or lower fungal derivatives comprising sources such as, but not limited to, Saccharomyces, Phaffia, Pichia, Mortierella, Alteroinonas, Pseuodoalteromonas, Pythium, Schizochytrium, Thraustochytrium, Ulkenia, and/or LC-PUFA containing bacteria such as Vibrio spp., and Shewanella spp.

Another embodiment of the invention is a feed or feed ingredient wherein the essential nutrients and oils normally provided by animal meal, fishmeal, and/or fish oil are replaced partially by macro- and/or microalgal biomass, macro- and/or mnicroalgal cells, or macro- and/or microalgal extracts plus additional supplementation with lower fungal sources such as, but not limited to, Saccharomyces, Phaffia, Pichia, Mortierella, Alteromonas, Pythium, Schizochiytrium, Thraustochytriumn, Ulkenia, and/or LC-PUFA-containing bacteria such as Vibrio spp. and Shewanella spp.

In another embodiment of the invention, a method is provided for production of a feed or feed ingredient that will replace the use of animal meal, fishmeal, or fish oil in feeds used for terrestrial or aquatic organisms wherein algae are added to the product to provide the essential nutrients and oils required for optimal growth.

In another embodiment of the invention, a method is provided for aquatic or terrestrial animal husbandry using a feed or feed ingredient wherein all animal products are eliminated and the feed contains microalgae, macroalgae, plants, and/or lower fungi such that the feed provides the essential nutrients and oils required for optimal growth.

The following examples are provided for exemplification purposes only and are not intended to limit the scope of the instant invention.

### EXAMPLES

### Example 1

Preparation of Macroalgal, Microalgal, Lower Fungal, and Bacterial Biomass.

Macroalgae, such as Ulva spp., Gracilaria spp. and Laminaria spp., are cultured in an open earthen pond using industrial grade nutrients to provide nitrogen, potassium and phosphorus elements. Algal thalli are harvested periodically, oven dried, then ground to a fine powder using standard methods. The thalli can also be ground wet to provide a fine slurry. Heterotrophic growth of macroalgal biomass is also a possibility (Durand et al. 1997).

Photosynthetic microalgae, such as Tetraselmis spp., Spirulina spp., Nannochloropsis spp., Navicula spp., and Chaetoceros spp., are cultured in enclosed bioreactors using FeCl₃, NaNO₃, and NaH₂PO₄ enriched f/2 medium (Guillard and Ryther 1962; Guillard 1975). Algae are harvested at stationary phase then concentrated by centrifugation, filtration, or flocculation. Algal pastes are dried (drum dried, spray dried, or the like) and ground into a fine powder.

Heterotrophic microalgae, such as Crypthecodinium spp., Chlorella spp. Haematococcus spp., Nitzschia spp.; lower fungi, such as Mortierella spp., or LC-PUFA containing bacteria such as Shewanella putrefaciens or Vibrio marinus are cultured in industrial fermentors using glucose as a source of energy and by following established culturing procedures (Boswell et al. 1992; Behrens and Kyle 1996). Microalgae are then harvested and centrifuged to produce a thick paste, dried (drum drying, spray drying, or the like) and ground into a fine powder. All algal sourced powders are homogenized in a specific proportion (dependant on animal species) and kept for later formulation with other feed ingredients.

### Example 2

Preparation of Grow-Out Diet for Sea Bream

Sea bream feed is formulated with the ingredients listed below using standard formulation methods (Lim and Sessa 1995). The feed is designed to include at least 45% protein, 13% lipids, and 0.5% DHA. Algal-based ingredients are produced as described in Example 1. In addition to proteins and lipids, the specific algal mix also provides essential nutrients for enhancing the fish growth. For example, Ulva sp. and Laminaria sp. are rich sources of polysaccharides and glycoproteins, Haematococcus sp., and Spirulina sp. are rich in carotenoids and antioxidants, while Crypthecodinium sp. and Mortierella sp. are rich in essential fatty acids (such as docosahexaenoic (DHA) and arachidonic acids (ARA)). The ingredient mix is then extruded to 3-10 mm pellet size using a standard pellet extruder.

TABLE 1
Diet composition for sea bream grow-out

| | |
|---|---|
| Algal Mix | 20% |
| Composition of Algal mixture: | |
| Ulva sp. | 10% |
| Spirulina platensis (a.k.a. Arthrospira platensis) | 5% |
| Crypihecodinium cohnii | 3% |
| Laminaria sp. | 1% |
| Haematococcus pluvialis | 1% |
| Fungal biomass (Mortierella alpina) | 1% |
| Soy protein concentrate | 56% |
| Wheat meal | 10% |
| Soy oil | 7.3% |
| Mineral mix | 1% |
| Lysine | 1% |
| Methionine | 0.5% |
| Glycine | 0.5% |
| Threonine | 0.2% |
| Vitamins mix | 1% |
| α-Tocopherol | 0.5% |
| Ascorbic acid | 0.5% |
| Betaine | 0.5% |

* Percentages are on dry weight basis. Final PUFA content is 0.6% DHA, 0.4% 16:4 + 18:4 (omega-3), and 0.15% ARA. Material sourcing: Soy protein concentrate, wheat meal and soy oil are obtained from Central Soya Company, Inc. Fort Wayne, IN. All trace minerals, vitamin mixes, and amino acids are obtained from A. Gresearch Inc. Joliet, IL and Bentoli, Inc. Homestead, FL. Fungal biomass (Mortierella alpina) is from Martek Biosciences.

### Example 3

Feeding of Sea Bream Fish.

Sea bream fingerlings at ca. 100 g size are stocked at 30 kg per m³ of seawater at a temperature of 25° C. Water quality is maintained by rapidly exchanging the tank water through mechanical and biofiltration systems. Fish are fed 4 times daily a total ration of 2% body weight and pellet size adjusted to fit the mouth opening of the growing fish. Experiment is terminated when fish reach an average commercial size of 400 g.

Daily growth rates are calculated according to the following formula: Growth rate=(Final average fish weight minus initial average weight)/n days.

Food conversion ratio (FCR) is calculated according to the following formula: FCR=Total food given/(total fish final biomass minus total fish initial biomass).

### Example 4

Preparation of Grow-Out Diet for Shrimp

Shrimp feed is formulated with the ingredients listed below using standard methods (Lim and Sessa 1995). The grow-out feed is designed to include at least 30% protein, 6% lipids, and 0.5% DHA and EPA. Algal-based ingredients are produced as described in Example I with the addition of diatoms (Chaetoceros sp. and Navicula sp.) for the required calcium and silica minerals in the shrimp diet. Tetraselmis sp. is also provided in the algal mix because of it provides critical components for the shrimp, such as eicosapentaenoic acid (EPA) and cholesterol. The ingredient mix is then extruded to 3-10 mm pellet size using a standard pellet extruder.

TABLE 2
Diet composition for grow-out diet for shrimp

| | |
|---|---|
| Algal Mix | 20% |
| Composition of Algal mixture: | |
| Ulva sp. | 5% |
| Spirulina platensis (or Arthrospira platensis) | 4% |
| Tetraselmis sp. | 3% |
| Chaetoceros sp. | 2% |
| Crypthecodinium cohnii | 2% |
| Navicula saprophila | 1% |
| Gracilaria sp. | 1% |
| Haematococcus pluvialis | 2% |
| Soy protein concentrate | 38% |
| Wheat meal | 33% |
| Soy oil | 4% |
| Mineral mix | 1% |
| Vitamins mix | 0.5% |
| α-Tocopherol | 0.5% |
| Ascorbic acid | 0.5% |
| Cholesterol | 0.5% |
| Betaine | 0.5% |
| Glycine | 0.5% |
| Lysine | 0.5% |
| Methionine | 0.5% |

* Percentages are on dry weight basis. Material sourcing as in Example 2. Final PUFA content is 0.54% DHA and EPA and 0.2% 16:4 + 18:4 (omega-3).

### Example 5

Feeding of Shrimp

Shrimp fry at ca. 10 g size are stocked at 10 kg per m³ of seawater at a temperature of 28° C. Water quality is maintained by rapidly exchanging the tank water through mechanical and biofiltration systems. Shrimp are fed 4 times daily a total ration of 2% body weight and pellet size adjusted to fit the mouth opening of the growing shrimp. The experiment is terminated when shrimp reach an average commercial size of 40 g. Daily growth rates and FCR are calculated as described in Example 3.

### Example 6

Preparation of Grow-Out Diet for Poultry

Broiler feed is formulated with the ingredients listed in Table 3 using standard methods. This feed is designed to include at least 25% protein, 16% lipids and 0.5% DHA. Algal-based ingredients are produced as described in Example 1. The ingredient mix is then pelleted to 0.5-3 mm pellet size using a standard pellet maker.

TABLE 3
Diet composition for poultry grow-out

| | |
|---|---|
| Algal Mix | 10% |
| Composition of Algal mixture: | |
| Ulva sp. | 4% |
| Spirulina platensis (or Arthrospira platensis) | 1% |
| Crypthecodinium cohnii | 3% |
| Laminaria sp. | 1% |
| Navicula sp. | 1% |
| Fungal biomass Mortierella sp. | 1% |
| Soy meal | 15% |
| Wheat grain | 24% |
| Corn grain | 15% |
| Alfalfa meal | 10% |
| Soy oil | 15% |
| Lime | 7% |
| Mineral mix | 1.5% |
| Vitamins mix | 0.5% |
| α-Tocopherol | 0.5% |
| Ascorbic acid | 0.5% |

* Percentages are on dry weight basis. Material sourcing as in Example 2. Final PUFA content is 0.64% DHAandEPA, 0.2% 16:4 + 18:4 (omega-3), and 0.15% ARA.

### Example 7

Feeding of Poultry

Broiler chickens at a size of ca. 100 g are housed in windowless sheds at a stocking density of 20 kg of bird weight per m². Temperature and ventilation are automatically controlled. Broilers are fed 4 times daily a total ration of 4% body weight and pellet size adjusted to fit the mouth opening of the growing chick. The experiment is terminated when broiler reaches an average commercial size of 2000 g. Daily growth rate and FCR are calculated as described in Example 3.

### Example 8

Preparation of Grow-Out Diet for Swine

Swine feed is formulated with the ingredients listed in Table 4 and designed to include at least 20% protein and 6% lipid (including 0.25% DHA).

TABLE 4
Diet composition for swine grow-out

| | |
|---|---|
| Algal Mix | 8% |
| Composition of Algal mixture: | |
| Ulva sp. | 4% |
| Spirulina platensis (or Arthrospira platensis) | 2% |
| Crypthecodinium cohnii | 2% |
| Fungal biomass Mortierella sp. | 1% |
| Soy protein (and/or pea protein ) | 15% |
| Wheat grain | 33.3% |
| Barley grain | 20% |
| Corn grain | 15% |
| Soy oil | 5% |
| Minerals mix | 2.5% |
| Trace element mix | 0.1% |
| Vitamins mix | 0.1% |

* Percentages are on dry weight basis. Material sourcing is the same as in Example 2. Final PUFA content is 0.4% DHA, 0.2% 16:4 + 18:4 (omega-3), and 0.15% ARA.

### Example 9

Feeding of Swine

Weaned piglets, 4 weeks old are housed in groups of 4 in straw-bedded pens with ad libitum access to diet and water. Upon reaching a commercial weight of 110 kg, pigs are weighed. Daily growth rate and FCR are calculated as described in Example 3.

### Example 10

Preparation of a Microalgal Diet for Shrimp

Shrimp feed is formulated to contain a vegetable protein source, a vegetable oil source, a vitamin and mineral premix, and a microalgal source of long chain polyunsaturated fatty acids. Such a composition is made using a mixture of 38% soy protein concentrate and 51% wheat meal as a protein source, 5% soy oil, 1% commercial mineral mix, 1% commercial vitamin mix, 0.5% alpha tocopherol, 0.5% ascorbic acid, 0.5% cholesterol, and 2.5% Crypthecodinium cohnii, as supplied by Martek Biosciences Corporation (Columbia, Md.). Other microalgal sources such as, but not limited to Schizochytrium sp., Ulkenia sp., Tetraselmis sp., Cyclotella sp. and etc., can be substituted for the C. cohnii as long as the total EPA and DHA levels are in excess of about 0.5%.

The ingredient mixture above is then prepared for use as a feed by extrusion into pellets of consumable size for the animals (typically 3-10 mm) using a standard extruder, or flake-dried using a rotary drum dryer. This feed is then provided to the animals as described in Example 5.

### Example 11

Macroalgal Diet for Shrimp

Shrimp feed is formulated to contain a vegetable protein source, a vegetable oil source, a vitamin and mineral premix, and a macroalgal source of long chain polyunsaturated fatty acids. Such a composition is made using a mixture of 38% soy protein concentrate and 44% wheat meal as a protein source, 3% flax oil, 2% soy oil, 1% commercial mineral mix, 1% commercial vitamin mix, 0.5% alpha tocopherol, 0.5% ascorbic acid, and 10% Laminaria. Other macroalgal sources such as, but not limited to Gracillaria, Ulva, brown seaweeds, red seaweeds, and etc., can be substituted for the Laminaria as long as the total omega-3 long chain polyunsaturated fatty acid (EPA and DHA) levels are in excess of about 0.5%.

The ingredient mixture above is then prepared for use as a feed by extruding into pellets of consumable size for the animals (typically 3-10 mm) using a standard extruder or flake dried using rotary drum dryer. This feed is then provided to the animals as described in Example 5.

### Example 12

Fungal Diet for Shrimp

Shrimp feed is formulated to contain a vegetable protein source, a vegetable oil source, a vitamin and mineral premix, and a fungal source of long chain polyunsaturated fatty acids. Such a composition is made using a mixture of 38% soy protein concentrate and 51% wheat meal as a protein source, 3% flax oil, 2% soy oil, 1% commercial mineral mix, 1% commercial vitamin mix, 0.5% alpha tocopherol, 0.5% ascorbic acid, 0.5% cholesterol, and 2.5% Mortierella alpina as supplied by Martek Biosciences Corporation (Columbia, Md.). Other fungal sources such as, but not limited to Pythium, Saprolegnia, Connidiobolus, Schizochytrium, Thraustochiytrium, and etc., can be substituted for the M. alpina as long as the total long chain polyunsaturated fatty acid levels (omega-3+Omega-6) are in excess of about 0.5%.

The ingredient mixture above is then prepared for use as a feed by extrusion into pellets of consumable size for the animals (typically 3-10 mm) using a standard extruder or flake-dried using rotary drum dryer. This feed is then provided to the animals as described in Example 5.

### Example 13

Microalgal/Fungal Diet for Shrimp

Shrimp feed is formulated to contain a vegetable protein source, a vegetable oil source, a vitamin and mineral premix, and a microalgal source of long chain polyunsaturated fatty acids and a fungal source of long chain polyunsaturated fatty acids. Such a composition is made using a mixture of 38% soy protein concentrate and 47% wheat meal as a protein source, 5% soy oil, 1% commercial mineral mix, 1% commercial vitamin mix, 0.5% alpha tocopherol, 0.5% ascorbic acid, and 3% Crypthecodinium cohnii as supplied by Martek Biosciences Corporation (Columbia, Md.) and 4% Mortierella alpina as supplied by Martek Biosciences Corporation (Columbia, Md.). Extracts of portions of the above algal and fungal sources can be substituted for the biomasses as long as the total EPA and DHA levels are in excess of about 0.5% and the total ARA levels are in excess of about 0.5%.

The ingredient mixture above is then prepared for use as a feed by extrusion into pellets of consumable size for the animals (typically 3-10 mm) using a standard extruder or flake-dried using rotary drum dryer. This feed is then provided to the animals as described in Example 5.

### Example 14

Microalgal/Macroalgal Diet for Shrimp

Shrimp feed is formulated to contain a vegetable protein source, a vegetable oil source, a vitamin and mineral premix, a microalgal source of long chain polyunsaturated fatty acids, and a fungal source of long chain polyunsaturated fatty acids. Such a composition is made using a mixture of 38% soy protein concentrate and 47% wheat meal as a protein source, 5% soy oil, 1% commercial mineral mix, 1% commercial vitamin mix, 0.5% alpha tocopherol, 0.5% ascorbic acid, 3% Crypthecodinium cohnii as supplied by Martek Biosciences Corporation (Columbia, Md.), and 5% Laminaria. Extracts of portions of the above algal and macroalgal sources can be substituted for the biomasses as long as the total EPA and DHA levels are in excess of about 0.5% and the total ARA levels are in excess of about 0.2%.

The ingredient mixture above is then prepared for use as a feed by extrusion into pellets of consumable size for the animals (typically 3-10 mm) using a standard extruder or flake-dried using a rotary drum dryer. This feed is then provided to the animals as described in Example 5.

### Example 15

Microalgal/Macroalgal/Fungal Diet for Shrimp

Shrimp feed is formulated to contain a vegetable protein source, a vegetable oil source, a vitamin and mineral premix, a microalgal source of long chain polyunsaturated fatty acids, and a fungal source of long chain polyunsaturated fatty acids. Such a composition is made using a mixture of 38% soy protein concentrate and 42% wheat meal as a protein source, 5% soy oil, 1% commercial mineral mix, 1% commercial vitamin mix, 0.5% alpha tocopherol, 0.5% ascorbic acid, 3% Crypthecodinium cohnii as supplied by Martek Biosciences Corporation (Columbia, Md.), 4% Mortierella alpina as supplied by Martek Biosciences Corporation (Columbia, Md.), and 5% Gracillaria. Extracts of portions of the above algal and fungal sources can be substituted for the biomasses as long as the total EPA and DHA levels are in excess of about 0.5% and the total ARA levels are in excess of about 0.5%.

The ingredient mixture above is then prepared for use as a feed by extrusion into pellets of consumable size for the animals (typically 3-10 mm) using a standard extruder or flake-dried using rotary drum dryer. This feed is then provided to the animals as described in Example 5.

### Example 16

Microalgal/Pea Protein Diet

Shrimp feed is formulated to contain a vegetable protein source, a vegetable oil source, a vitamin and mineral premix, and a microalgal source of long chain polyunsaturated fatty acids. Such a composition is made using a mixture of 38% soy protein concentrate and 50% pea meal as a protein source, 5% soy oil, 1% commercial mineral mix, 1% commercial vitamin mix, 0.5% alpha tocopherol, 0.5% ascorbic acid, 0.5% cholesterol, and 3.5% Crypthecodinium cohnii as supplied by Martek Biosciences Corporation (Columbia, Md.). Other long chain polyunsaturated fatty acid sources such as, but not limited to Schizochytrium sp., Ulkenia sp., Tetraselmis sp., Cyclotella sp. etc., can be substituted for the C. cohnii while maintaining the total EPA and DHA levels in excess of about 0.5%.

The ingredient mixture above can then be prepared for use as a feed by extruding into pellets of consumable size for the animals (typically 3-10 mm) using a standard extruder or flake-dried using a rotary drum dryer. This feed is then provided to the animals as described in Example 5.

### Example 17

Microalgal/Protein Hydrolysate Diet

Shrimp feed is formulated to contain a vegetable protein source, a vegetable oil source, a vitamin and mineral premix, and a microalgal source of long chain polyunsaturated fatty acids. Such a composition is made using a mixture of 88% soy protein concentrate, 5% soy oil, 1% commercial mineral mix, 1% commercial vitamin mix, 0.5% alpha tocopherol, 0.5% ascorbic acid, 0.5% cholesterol, and 3.5% Crypthecodinium cohnii as supplied by Martek Biosciences Corporation (Columbia, Md.). Other long chain polyunsaturated fatty acid sources such as, but not limited to, Schizochytrium sp., Ulkenia sp., Tetraselmis sp., Cyclotella sp., and etc., can be substituted for the C. cohnii, maintaining the total EPA and DHA levels in excess of about 0.5%.

The ingredient mixture above is then prepared for use as a feed by extruding into pellets of consumable size for the animals (typically 3-10 mm) using a standard extruder or flake dried using rotary drum dryer. This feed is then provided to the animals as described in Example 5.

### Example 18

Macroalgal Diet with Fishmeal

Shrimp feed is formulated to contain a small amount of fishmeal, a vegetable protein source, a vegetable oil source, a vitamin and mineral premix, and a microalgal source of long chain polyunsaturated fatty acids. Such a composition is made using 4% fishmeal, a mixture of 38% soy protein concentrate, and 47.5% wheat meal as a protein source, 5% soy oil, 1% commercial mineral mix, 1% commercial vitamin mix, 0.5% alpha tocopherol, 0.5% ascorbic acid, 0.5% cholesterol, and 2.5% Crypthecodinium cohnii, as supplied by Martek Biosciences Corporation (Columbia, Md.). Other microalgal sources such as, but not limited to, Schizochytrium sp., Ulkenia sp., Tetraselnis sp., Cyclotella sp., and etc., can be substituted for the C. cohnii, while maintaining the total EPA and DHA levels in excess of about 0.5%.

This ingredient mixture is then prepared for use as a feed by extrusion into pellets of consumable size for the animals (typically 3-10 mm) using a standard extruder or flake-dried using rotary drum dryer. This feed is then provided to the animals as described in Example 5.

### Example 19

Fungal Diet with Fishmeal

Shrimp feed is formulated to contain a small amount of fishmeal, a vegetable protein source, a vegetable oil source, a vitamin and mineral premix, and a fungal source of long chain polyunsaturated fatty acids. Such a composition is made using 4% fish oil, a mixture of 38% soy protein concentrate, and 46% wheat meal as a protein source, 3% flax oil, 2% soy oil, 1% commercial mineral mix, 1% commercial vitamin mix, 0.5% alpha tocopherol, 0.5% ascorbic acid, 0.5% cholesterol, and 4% Mortierella alpina as supplied by Martek Biosciences Corporation (Columbia, Md.). Other fungal sources such as, but not limited to, Pythium, Saprolegnia, Connidiobolus, Schizochytrium, Thraustochytrium, and etc., can be substituted for the M. alpina while maintaining the total long chain polyunsaturated fatty acid levels in excess of about 0.5%.

The ingredient mixture above is then prepared for use as a feed by extrusion into pellets of consumable size for the animals (typically 3-10 mm) using a standard extruder or flake-dried using a rotary drum dryer. This feed is then provided to the animals as described in Example 5.

### Example 20

Microalgal Diet with Pea Protein and Fishmeal

Shrimp feed is formulated to contain a small amount of fishmeal, a vegetable protein source, a vegetable oil source, a vitamin and mineral premix, and a microalgal source of long chain polyunsaturated fatty acids. Such a composition is made using 4% fish meal, a mixture of 38% soy protein concentrate and 47% pea meal as a protein source, 5% soy oil, 1% commercial mineral mix, 1% commercial vitamin mix, 0.5% alpha tocopherol, 0.5% ascorbic acid, 0.5% cholesterol, and 3% Crypthecodinium cohnii as supplied by Martek Biosciences Corporation (Columbia, Md.). Other microalgal sources such as, but not limited to, Schizochytrium sp., Ulkenia sp., Tetraselmis sp., Cyclotella sp. and etc., can be substituted for the C. cohnii while maintaining the total EPA and DHA levels in excess of about 0.5%.

The ingredient mixture above is then prepared for use as a feed by extruding into pellets of consumable size for the animals (typically 3-10 mm) using a standard extruder or flake dried using rotary drum dryer. This feed is then provided to the animals as described in Example 5.

### Example 21

Preparation of a High DHA Microalgal Diet for Shrimp

Shrimp feed is formulated to contain a vegetable protein source, a vegetable oil source, a vitamin and mineral premix, and a microalgal source of long chain polyunsaturated fatty acids. Such a composition is made using a mixture of 38% soy protein concentrate and 43.5% wheat meal as a protein source, 5% soy oil, 1% commercial mineral mix, 1% commercial vitamin mix, 0.5% alpha tocopherol, 0.5% ascorbic acid, 0.5% cholesterol, and 10% Crypthecodinium cohnii as supplied by Martek Biosciences Corporation (Columbia, Md.). Other microalgal sources such as, but not limited to, Schizochytrium sp., Ulkenia sp., Tetraselmis sp., Cyclotella sp., and etc., can be substituted for the C. cohnii while maintaining the total EPA and DHA levels in excess of about 0.5%.

The ingredient mixture above is then prepared for use as a feed by extruding into pellets of consumable size for the animals (typically 3-10 mm) using a standard extruder or flake dried using rotary drum dryer. This feed is then provided to the animals as described in Example 5. The DHA content of the above feed is 2% by weight and it is used as a broodstock diet or a finishing diet for shrimp.

### Example 22

Preparation of a High DHA Microalgal Diet for Shrimp

Shrimp feed is formulated to contain a vegetable protein source, a vegetable oil source, a vitamin and mineral premix, and a microalgal source of long chain polyunsaturated fatty acids. Such a composition is made using a mixture of 38% soy protein concentrate and 28.5% wheat meal as a protein source, 5% soy oil, 1% commercial mineral mix, 1% commercial vitamin mix, 0.5% alpha tocopherol, 0.5% ascorbic acid, 0.5% cholesterol, and 25% Crypthecodinium cohnii as supplied by Martek Biosciences Corporation (Columbia, Md.). Other microalgal sources such as, but not limited to, Schizochytrium sp., Ulkenia sp., Tetraselmis sp., Cyclotella sp. and etc., can be substituted for the C. cohnii, while maintaining the total EPA and DHA levels in excess of about 0.5%.

This ingredient mixture is then prepared for use as a feed by extruding into pellets of consumable size for the animals (typically 3-10 mm) using a standard extruder or flake-dried using rotary drum dryer. This feed is then provided to the animals as described in Example 5. The DHA content of this feed is 5% by weight and is used as a finishing diet for shrimp.

### Example 23

Microalgal/Macroalgal Diet for Salmonids

Sahnonid (e.g., salmon & trout) feed is formulated to contain a vegetable protein source, a vegetable oil source, a vitamin and mineral premix, and a microalgal source of long chain polyunsaturated fatty acids and a fungal source of long chain polyunsaturated fatty acids. Such a composition is made using a mixture of 23.6% pea protein concentrate, 10% wheat, 5% wheat gluten, and 25% soy protein SPF as protein sources, 25% soy oil, 0.4% commercial mineral mix, 0.2% commercial vitamin mix, 0.5% alpha tocopherol, 0.2% ascorbic acid, amino acids (0.5% lysine, 0.2% methionine, 0.2% threonine, and 0.2% Betaine), and 9% algal mixture (5% Ulva, 3% Crypthecodinium cohnii as supplied by Martek Biosciences Corporation (Columbia, Md.) and 4% Haematococcus as supplied by Cyanotech Corporation (Kona, Hi.)). Extracts of portions of the above algal sources can be substituted for the biomasses as long as the total EPA and DHA levels are in excess of about 0.5% and the total ARA levels are in excess of about 0.5%.

This ingredient mixture is then prepared for use as a feed by extrusion into pellets of consumable size for the fish (typically 3-10 mm) using a standard extruder or flake-dried using rotary drum dryer. This feed is then provided to the animals as described in Example 3 for sea bream.

### Example 24

Shrimp Diet Containing Microalgal Components

A shrimp diet was prepared using poultry by-product meal, a vegetable protein source, a vegetable oil source, a vitamin and mineral premix, and a DHA-containing microalgal biomeal. The poultry by-product meal comprised 40% Profound® (AF Protein Inc), the vegetable protein source comprised 30% soy meal; the vegetable oil comprised 1.5% soy oil and 1.2% flax oil; and the DHA-containing microalgal biomeal comprised 2% solvent-extracted Crypthecodinium cohnii (Martek Biosciences Corp, Columbia, Md.). Other DHA-containing biomeals such as, but not limited to, solvent-extracted chytrids such as Schizochytrium sp., Thraustochytrium sp., and Ulkenia sp., and solvent extracted diatoms such as Tetraselmis sp. and Cyclotella sp. biomeal can be supplemented in this composition at levels from 0.5% to 50% of the total weight of the feed.

This ingredient mixture was then prepared for use as a feed by extrusion into pellets of consumable size for the animals (typically 3-10 mm) using a standard extruder. A rotary drum dryer is also suitable for this task. This feed was then provided to the animals as described in Example 5.

### Example 25

Shrimp Diet Containing Microalgal Components

A shrimp diet was prepared using poultry by-product meal, a vegetable protein source, a vegetable oil source, a vitamin and mineral premix, and a microbial source of DHA and ARA (Table 1). The poultry by-product meal comprised 40% Profound® (AF Protein Inc), the vegetable protein source comprised 30% soy meal, the vegetable oil comprised 1.5% soy oil and 1.2% flax oil, the microbial DHA source comprised 2% Schizochiytrium biomass (martek Biosciences Corp, Columbia, Md.), and the microbial ARA source comprised 0.5% AquaGrow® ARA (Advanced BioNutrition Corp, Columbia, Md.).

TABLE 5
Composition of test diets for fishmeal replacement strategy

| Ingredient | Diet 1 (%) | Diet 2 (%) | Diet 3 (%) |
|---|---|---|---|
| Profound (AF Protein) | 39.00 | 39.00 | 39.00 |
| Soybean meal | 29.50 | 30.20 | 30.50 |
| Schizochytrium DHA | 2.00 | 0.50 | 0.00 |
| AquaGrow ARA | 0.50 | 0.13 | 0.00 |
| Fish oil (Menhaden) | 0.00 | 0.00 | 3.04 |
| Soy oil | 1.47 | 1.53 | 0.00 |
| Flax oil | 0.48 | 1.23 | 0.00 |
| Wheat starch | 1.98 | 2.34 | 2.39 |
| Whole wheat | 20.00 | 20.00 | 20.00 |
| Trace mineral premix | 0.50 | 0.50 | 0.50 |
| Vitamin premix | 1.80 | 1.80 | 1.80 |
| Choline chloride | 0.20 | 0.20 | 0.20 |
| Stay C 250 mg/kg 14 | 0.07 | 0.07 | 0.07 |
| CaP-dibasic | 2.00 | 2.00 | 2.00 |
| Lecithin | 0.50 | 0.50 | 0.50 |
| Total | 100.00 | 100.00 | 100.00 |

The ingredient mixture above was then prepared for use as a feed by extrusion into pellets of consumable size for the animals (typically 1-10 mm) using a standard extruder or flake dried using rotary drum dryer. This feed was provided to the animals on a daily basis and growth rate was measured over the course of 12 weeks. The data provided in Table 6 indicates that there were little or no differences in the growth and final weight and survival of shrimp fed with the fish oil/fishmeal replacement diets relative to a standard diet containing 35% fish meal and 5% fish oil (Rangen Control Diet).

TABLE 6
Shrimp weights and survival following 16 weeks growth
with different diets replacing fish meal (Diets
1-3) and a control (Rangen shrimp) diet

| | Diet 1 | Diet 2 | Diet 3 | Control |
|---|---|---|---|---|
| Mean Weight (g) | 17.10 | 17.89 | 17.02 | 18.50 |
| 95% CL | 1.41 | 0.51 | 1.09 | 1.30 |
| Survival | 1.30 | 1.36 | 1.59 | 1.43 |
| 95% CL | 0.22 | 0.41 | 0.51 | 0.24 |

### Example 26

Complete Vegetable-Based Diet for Shrimp

A shrimp diet is prepared using a mixture of vegetable protein sources, a vegetable oil source, a vitamin and mineral premix, and a microbial source of DHA and ARA (Table 3). The vegetable protein mixture comprises 58% soy meal, 10% pea meal, and 9% corn gluten; the vegetable oil comprises 1.5% soy oil and 2% flax oil; the microbial DFIA source comprises 0.5% Schizochytrium biomass (Martek Biosciences Corp, Columbia, Md.); and the microbial ARA source comprises 0.13% AquaGrow ARA (Advanced BioNutrition Corp, Columbia, Md.). Other microbial DHA-containing material may include chytrids such as Thraustochytrium sp., and Ulkenia sp., and algae such as Crypthecodinium sp., Tetraselmis sp. and Cyclotella sp.

TABLE 7
Composition of test diet for total fishmeal replacement
using vegetable and microbial products

| Ingredient (%) | Diet 1 |
|---|---|
| Soybean meal | 58.10 |
| Pea meal | 10.00 |
| Corn gluten meal | 9.00 |
| Schizochytrium DHA | 0.50 |
| AquaGrow ARA | 0.13 |
| Soy oil | 0.20 |
| Flax oil | 2.00 |
| Whole wheat | 14.00 |
| Trace mineral premix | 0.50 |
| Vitamin premix | 1.80 |
| Choline chloride | 0.20 |
| Stay C 250 mg/kg 14 | 0.07 |
| CaP-dibasic | 2.00 |
| Lecithin | 0.50 |
| Betaine-3DP | 0.50 |
| Total: | 100.00 |

The ingredient mixture above is then prepared for use as a feed by extruding into pellets of consumable size for the animals (typically 1-10 mm) using a standard extruder or flake-dried using rotary drum dryer.

### Example 27

Complete Organic Vegetable-Based Diet for Shrimp

A shrimp diet was prepared using a mixture of vegetable protein sources, a vegetable oil source, a vitamin and mineral premix, and a microbial source of DHA and ARA as in Example 25, all of which have been certified as organic. The vegetable protein mixture comprised 58% soy meal, 10% pea meal, and 9% corn gluten; the vegetable oil comprised 1.5% soy oil and 2% flax oil; the microbial DHA source comprised 0.5% Schizochytrium biomass (Martek Biosciences Corp, Columbia, Md.) and the microbial ARA source comprised 0.13% AquaGrow ARA (Advanced BioNutrition Corp, Columbia, Md.). Other microbial DHA-containing material is also suitable, including, for example, chytrids such as Thraustochytrium sp., and Ulkenia sp., and algae such as Crypthecodinium sp., Tetraselmis sp. and Cyclotella sp.

The ingredient mixture above was then prepared for use as a feed by extruding into pellets of consumable size for the animals (typically 1-10 mm) using a standard extruder. Flake drying using a rotary drum dryer in a facility that has been certified as one capable of producing organic products is also suitable. The resulting feed is certifiable as “Organic” under the USDA definitions of an Organic Product. Feeding of shrimp using organic farming practices and the organic feed described in this example allow the shrimp so produced to be labeled as “Organic Shrimp” .

### Example 28

A Fishmeal Substitute Comprising EPA/DHA-Containing and ARA-Containing Plant Material for an Animal Diet

As a replacement for fishmeal in an animal feed or feed additive, certain plant materials containing DHA and ARA can be used. Examples of plant material (not including algae) containing EPA/DHA would include certain mosses (e.g., Physcomitrella patens, Rhytidiadelphus squarrosus, or Ceratodon purpureus) or genetically engineered plant species producing DHA (e.g., as described in U.S. Pat. No. 6,677,145, U.S. Pat. No. 6,635,451, or U.S. Application No. 20030101486). Examples of plant material (not including algae) containing ARA would include certain mosses (e.g., Physcomitrella patens) or genetically engineered plant species producing ARA (e.g., as described in U.S. Pat. No. 6,677,145, U.S. Pat. No. 6,635,451).

A shrimp feed or feed additive is prepared using a mixture of vegetable protein sources, a vegetable oil source, a vitamin and mineral premix, and a plant source of DHA and ARA (chosen from the examples above). The vegetable protein mixture comprises 58% soy meal, 10% pea meal, and 9% corn gluten; the vegetable oil comprises 1.5% soy oil and 2% flax oil; the plant DHA source comprises 5% Physcomitrella lipid, and the plant ARA source comprises 2% modified brassica oil containing 30% ARA (Abbott Labs).

The totally vegetarian ingredient mixture above is-then prepared for use as a feed or feed additive by extruding into pellets of consumable size for the animals (typically 1-10 mm) using a standard extruder or flake dried using rotary drum dryer using conventional manufacturing practices. This totally vegetarian feed is then provided to shrimp using a standard feeding regimen well known to those in the industry for the growth of shrimp.

A salmon feed or feed additive is prepared using poultry by-product meal, a vegetable protein source, a vegetable oil source, a vitamin and mineral premix, and a plant source of DHA and ARA (chosen from the examples above). The poultry by-product meal comprises 40% Profound® (AF Protein Inc), the vegetable protein source comprises 30% soy meal; the vegetable oil comprises 1.5% soy oil and 1.2% flax oil; the plant DHA source comprises 2% moss and the plant ARA source comprises 30% modified soy oil (Abbott Labs).

The plant DHA/poultry by-product-containing ingredient mixture above is then prepared for use as a salmon feed or feed additive by extruding into pellets of consumable size for the animals (typically 1-10 mm) using a standard extruder or flake dried using rotary drum dryer using conventional manufacturing practices. This feed or feed additive is then provided to salmon using a standard feeding regimen well known to those in the industry for the growth of salmon.

### REFERENCES

The specification is most thoroughly understood in light of the following references, all of which are hereby incorporated by reference in their entireties.
1. Abe T, Nakagawa A, Higuchi H, Yamanaka T (1998) Process of feeding juvenile fish with astaxanthin-containing zooplankton. In. Kyowa Hakko Kogyo Co., Ltd.
2. Adey WH, Purgason R (1998) Animal feedstocks comprising harvested algal turf and a method of preparing and using the same. In: PAT 02-10-98 05715774 NDN-095-0259-5057-0. Aquatic BioEnhancement Systems, USA
3. Allen V, Pond K (2002) Seaweed supplement diet for enhancing immune response in mammals and poultry. In: U.S. Pat. No. 6,338,856 B1. Texas Tech Univ., USA.
4. Allen V, Pond K, Saker K, Fonetont J (2002) Seaweed supplement diet for enhancing immune response in mammals and poultry. In: U.S. Pat. No. 6,342,242 B1. Texas Tech Univ. & Virginia Tech Intellectual Properties, Inc.
5. Behrens P W, Kyle D J (1996) Microalgae as a source of fatty acids. J Food Lipids 3:259-272.
6. Boswell K D B, Gladue R, Prima B, Kyle D J (1992) SCO production by fermentive microalgae. In: Kyle D J, Ratledge C (eds) Industrial Applications of Single Cell Oils. American Oil Chemists Society, Champaign. Ill., pp 274-286.
7. De Rosa S et al. (2001) Chemical composition and biological activities of the Black Sea algae Polysiphonia denudata (Dillw.) Kutz. and Polysiphonia denudata f. fragilis (Sperk) Woronich. Z Naturforsch [C] 56:1008-1014.
8. Durand M et al. (1997) Fermentation of green alga sea-lettuce (Ulva sp) and metabolism of its sulphate by human colonic microbiota in a semi-continuous culture system. Reprod Nutr Dev 37:267-283.
9. Faulkner D J (2002) Marine natural products. Nat Prod Rep 19:1-48.
10. Ginzberg A, Cohen M, Sod-Moriah U A, Shany S, Rosenshtrauch-A, Arad S M (2000) Chickens fed with biomass of the red microalga Porphyridium sp. have reduced blood cholesterol level and modified fatty acid composition in egg yolk. J Appl Phycol 12:325-330.
11. Gonzalez R, Ledon N, Remirez D (2002) Role of histamine in the inhibitory effects of phycocyanin in experimental models of allergic inflammatory response. Mediators of Inflammation 11:81-85.
12. Grinstead G, Tokach M, Dritz S, Goodband R, Nelssen J (2000) Effects of Spirulina platensis on growth performance of weanling pigs. Animal Feed Sci Technol 83:237-247.
13. G S G, M D T, S S D, R D G, J L. N (2000) Effects of Spirulina platensis on growth performance of weanling pigs. Animal Feed Sci Technol 83:237-247.
14. Guillard R R L (1975) Culture of phytoplankton for feeding marine invertebrates. In: Smith W L, Chanley M H (eds) Culture of Marine Invertebrate Animals. Plenum Press, New York, USA, pp 26-60.
15. Guillard R R L, Ryther J H (1962) Studies of marine planktonic diatoms. I. Cyclotella nana Hustedt and Detonula confervacea Cleve. Can J Micro 8:229-239.
16. He M L, Hollwich W, Rambeck W A (2002) Supplementation of algae to the diet of pigs: a new possibility to improve the iodine content in the meat. J Animal Physiol Animal Nutri 86:97-104.
17. Hellio C, De La Broise D, Dufosse L, Le Gal Y, Bourgougnon N (2002) Inhibition of marine bacteria by extracts of macroalgae: potential use for environmentally friendly antifouling paints. Mar Enivron Res 52:231-247.
18. Jones A (1988) Algal extracellular products-antimicrobial substances. In: Rogers L, Gallon J (eds) Biochemistry of the algae and cyanobacteria. Claredon Press, Oxford, pp 256-281.
19. Kumvan W, Kaew K, Butryee C, Kupradinun P, Kusamran W R, Tepsuwan A (2001) Antigenotoxic and anticlastogenic effects of Porphyra spp. Mutation Res 483:S112.
20. Kyle D J, Reeb S E, Sicotte V J (1998) Dinoflagellate biomass, methods for its production, and compositions containing the same. In. Martek Biosciences Corporation.
21. Lim C, Sessa D (1995) Nutrition and Utilization Technology in Aquaculture. AOCS Press, Champaign, Ill.
22. Lu J, Yoshizaki G, Sakai K, Takeuchi T (2002) Acceptability of raw Spirulina platensis by larval tilapia Oreochromis niloticus. Fisheries Sci 68:51-58.
23. Mason C, Gleason F (1981) An antibiotic from Scytonema hofmanni cyanophyta. J Phycol 17.
24. Metting B, Pyne J (1986) Biologically active compounds from microalgae. Enzyme Microb. Technol. 8:386-394.
25. Minton J E, Dritz S S, Higgins J J, Turner J L (2002) Effects of Ascophyllum nodosum extract on growth performance and immune function of young pigs challenged with Salmonella typhimurium. J Animal Sci 80:1947-1953.
26. Neves S A, Dias-Baruffi M, Freitas A L P, Roque-Barreira M C (2001) Neutrophil migration induced in vivo and in vitro by marine algal lectins. Inflammation Research 50:486-490.
27. Oufdou K, Mezrioui N, Oudra B, Loudiki M, Barakate M, Sbiyy.a B (2001) Bioactive compounds from Pseudanabaena species (Cyanobacteria). Microbios 106:21-29.
28. Piccardi R et al. (2002) Potential applications in agriculture of extracts and biomass of Nostoc sp. ATCC 53789. In: Int. Applied Phycology Society, Spain.
29. Prati M, Molteni M, Pomati F, Rossetti C, Bernardini G (2002) Biological effect of the Planktothrix sp. FP1 cyanobacterial extract. Toxicon 40:267-272.
30. Seya T, Hazeki K, Hirahashi T, Matsumoto MS, Yoshiko; Ui, Michio (2002) Activation of the human innate immune system by Spirulina: Augmentation of interferon production and NK cytotoxicity by oral administration of hot water extract of Spirulina platensis. Intl Immunopharmacology 2:423-434.
31. Simopoulos AP (1999) New products from the agri-food industry: the return of n-3 fatty acids into the food supply. Lipids 34 Suppl:S297-301.
32. Tan M I, Siddiq A S, Y.; Barlian, A.; Haga, S. (2002) Effect of antitumor activity of Sargassum siliquosum on breast cancer cell line T47D. In Vitro Cellular & Developmental Biology Animal 38:8A.

1-59. (canceled)

60. An animal feed comprising not more than 5% animal-derived materials by dry weight, the feed comprising one or more ingredients selected from the group consisting of
i) macroalgae-derived materials,
ii) microalgae-derived materials,
iii) lower fungus-derived materials, and
iv) plant-derived materials that comprise at least one of docosahexaenoic acid (DHA), eicosapentaenoic acid (EPA), and arachidonic acid (ARA).

61. The feed of claim 60, wherein the feed is free of animal-derived materials.

62. The feed of claim 61, wherein the feed comprises a macroalgae-derived material.

63. The feed of claim 62, wherein the feed comprises from about 0.1% to about 30% macroalgae-derived materials by dry weight.

64. The feed of claim 62, wherein the macroalgae-derived material comprises a bioactive compound.

65. The feed of claim 64, wherein the feed contains the bioactive compound in an amount effective to effect at least one of immunoenhancement, growth promotion, disease resistance, antiviral action, antibacterial action, improved gut function, probiont colonization stimulation, improved food conversion, improved reproductive performance, improved coat, and improved skin in the animal.

66. The feed of claim 61, wherein the feed comprises a microalgae-derived material.

67. The feed of claim 66, wherein the feed comprises from about 0.1% to about 30% microalgae-derived materials by dry weight.

68. The feed of claim 66, wherein the microalgae-derived material comprises a bioactive compound.

69. The feed of claim 68, wherein the feed contains the bioactive compound in an amount effective to effect at least one of immunoenhancement, growth promotion, disease resistance, antiviral action, antibacterial action, improved gut function, probiont colonization stimulation, improved food conversion, improved reproductive performance, improved coat, and improved skin in the animal.

70. The feed of claim 61, wherein the feed comprises a lower fungus-derived material.

71. The feed of claim 70, wherein the feed comprises from about 0.1% to about 30% lower fungus-derived materials by dry weight.

72. The feed of claim 70, wherein the lower fungus-derived material comprises a bioactive compound.

73. The feed of claim 72, wherein the feed contains the bioactive compound in an amount effective to effect at least one of immunoenhancement, growth promotion, disease resistance, antiviral action, antibacterial action, improved gut function, probiont colonization stimulation, improved food conversion, improved reproductive performance, improved coat, and improved skin in the animal.

74. The feed of claim 60, wherein the feed comprises at least one of DHA and EPA in an amount, either individually or in combination, from about 0.25% to about 5.0% by dry weight.

75. The feed of claim 60, wherein the feed comprises an animal-derived material in an amoung from about 1% to about 5% by dry weight.

76. The feed of claim 60, wherein the feed comprises poultry by-product meal.

77. The feed of claim 60, wherein the feed comprises a genetically modified plant-derived material.

78. A method of preparing an animal feed, the method comprising combining one or more ingredients selected from the group consisting of
i) macroalgae-derived materials,
ii) microalgae-derived materials,
iii) lower fungus-derived materials, and
iv) plant-derived materials that comprise at least one of docosahexaenoic acid (DHA), eicosapentaenoic acid (EPA), and arachidonic acid (ARA),
in amount sufficient to achieve a content of at least one of DHA and EPA, either individually or
in combination, from about 0.25% to about 5.0% by dry weight of the feed, wherein the resulting feed does not include more than 5% animal-derived materials by dry weight.

79. The method of claim 78, wherein the feed is free of animal-derived materials.

80. A method of raising an animal, the method comprising feeding the animal a complete animal feed comprising not more than 5% animal-derived materials by dry weight, the feed comprising one or more ingredients selected from the group consisting of
i) macroalgae-derived materials,
ii) microalgae-derived materials,
iii) lower fungus-derived materials, and
iv) plant-derived materials that comprise at least one of docosahexaenoic acid (DHA), eicosapentaenoic acid (EPA), and arachidonic acid (ARA).

81. The method of claim 80, wherein the feed is free of animal-derived materials.
